(19) United States
(12) Patent Application Publication
(10) Pub. No.: US 2022/0243341 A1
(43) Pub. Date: Aug. 4, 2022

(54) CO2 CONVERSION WITH NANOWIRE-NANOPARTICLE ARCHITECTURE

(71) Applicant: The Regents of the University of Michigan, Ann Arbor, MI (US)

(72) Inventors: Baowen Zhou, Ann Arbor, MI (US); Xianghua Kong, Montreal (CA); Hong Guo, Montreal (CA); Zetian Mi, Ann Arbor, MI (US)

(21) Appl. No.: 17/629,679

(22) PCT Filed: Jul. 24, 2020
(86) PCT No.: PCT/US2020/043449

§ 371 Date: Jan. 24, 2022

Related U.S. Application Data
(60) Provisional application No. 62/878,607, filed on Jul. 25, 2019.

(51) Int. Cl. C25B 11/037; C25B 11/089; C25B 11/077; C25B 11/059; C25B 11/054; C25B 9/50

(52) U.S. Cl. CPC C25B 11/037; C25B 11/089; C25B 11/077; C25B 3/21; C25B 11/054; C25B 9/50; C25B 11/059

(57) ABSTRACT

An electrode of a chemical cell includes a substrate having a surface, an array of conductive projections supported by the substrate and extending outward from the surface of the substrate, each conductive projection of the array of conductive projections having a semiconductor composition for catalytic conversion of carbon dioxide (CO₂) in the chemical cell, and a plurality of nanoparticles disposed over the array of nanowires, each nanoparticle of the plurality of nanoparticles having a metallic composition for the catalytic conversion of CO₂ in the chemical cell. Each nanoparticle of the plurality of nanoparticles has a size at least an order of magnitude smaller than a lateral dimension of each conductive projection of the array of conductive projections.

### CROSS-REFERENCE TO RELATED APPLICATION

This application claims the benefit of U.S. provisional application entitled “CO₂ Conversion with Nanowire-Nanoparticle Architecture,” filed Jul. 25, 2019, and assigned Ser. No. 62/878,607, the entire disclosure of which is hereby expressly incorporated by reference.

### BACKGROUND OF THE DISCLOSURE

### Field of the Disclosure

The disclosure relates generally to photoelectrochemical and other chemical conversion of carbon dioxide (CO₂) into formic acid.

### Brief Description of Related Technology

Photoelectrochemical (PEC) reduction of carbon dioxide (CO₂) with water (H₂O) into fuels and chemicals, so-called artificial photosynthesis, is a promising strategy for storing intermittent solar energy and alleviating anthropogenic carbon emissions. Among a vast variety of CO₂ reduction products, formic acid (HCOOH) is an energy-dense liquid fuel and very useful chemical in industry. The conversion to formic acid requires only a two-electron transfer, and therefore is kinetically favorable to produce relative to other complex products, such as CH₃OH, CH₄, C₂H₄, and C₂H₅OH. However, efficient and selective photoelectrochemical reduction of CO₂ to HCOOH with large turnover frequency (TOF) at low overpotential still remains a substantial challenge due to the chemical inertness of CO₂, the complex reaction network of CO₂ conversion, and the severe competition of hydrogen evolution.

Photocathodes having a semiconductor light absorber and electrocatalysts have been used for artificial photosynthesis of HCOOH from CO₂ reduction. Various electrocatalysts, such as molecular complexes, enzymes, and metals (e.g., Pb, In, Cu, and Sn) in conjunction with various semiconductors, have been developed for CO₂ to HCOOH transformation. In spite of some notable achievements, the efficiency of these photoelectrodes remains far from any practical application due to the low sunlight-harvesting efficiency, sluggish charge carrier extraction, low atom-utilization efficiency, and ineffective CO₂ activation.

### SUMMARY OF THE DISCLOSURE

In accordance with one aspect of the disclosure, an electrode of a chemical cell includes a substrate having a surface, an array of conductive projections supported by the substrate and extending outward from the surface of the substrate, each conductive projection of the array of conductive projections having a semiconductor composition for catalytic conversion of carbon dioxide (CO₂) in the chemical cell, and a plurality of nanoparticles disposed over the array of conductive projections, each nanoparticle of the plurality of nanoparticles having a metallic composition for the catalytic conversion of carbon dioxide (CO₂) in the chemical cell. Each nanoparticle of the plurality of nanoparticles has a size at least an order of magnitude smaller than a lateral dimension of each conductive projection of the array of conductive projections.

In accordance with another aspect of the disclosure, a photocathode for a photoelectrochemical cell includes a substrate including a light absorbing material, the light absorbing material being configured to generate charge carriers upon solar illumination, an array of nanowires supported by the substrate, each nanowire of the array of nanowires being configured to extract the charge carriers from the substrate, each nanowire of the array of nanowires including gallium nitride, and a plurality of nanoparticles distributed across each nanowire of the array of nanowires, each nanoparticle of the plurality of nanoparticles having a metallic composition for the catalytic conversion of carbon dioxide (CO₂) in the photoelectrochemical cell into formic acid. Each nanoparticle of the plurality of nanoparticles has a size at least an order of magnitude smaller than a lateral dimension of each nanowire of the array of nanowires.

In accordance with yet another aspect of the disclosure, a photocathode for a photoelectrochemical cell includes a substrate including a light absorbing material, the light absorbing material being configured to generate charge carriers upon solar illumination, an array of nanowires supported by the substrate, each nanowire of the array of nanowires being configured to extract the charge carriers from the substrate, each nanowire of the array of nanowires including gallium nitride, and a plurality of nanoparticles distributed across each nanowire of the array of nanowires, each nanoparticle of the plurality of nanoparticles including tin for the catalytic conversion of carbon dioxide (CO₂) in the photoelectrochemical cell into formic acid.

In accordance with still another aspect of the disclosure, a method of fabricating an electrode of an electrochemical system includes growing an array of nanowires on a semiconductor substrate, each nanowire of the array of nanowires having a semiconductor composition for catalytic conversion of carbon dioxide (CO₂) in the electrochemical system, and depositing a plurality of nanoparticles across each nanowire of the array of nanowires, each nanoparticle of the plurality of nanoparticles having a metallic composition for the catalytic conversion of carbon dioxide (CO₂) in the electrochemical system. Depositing the plurality of nanoparticles includes implementing a number of electrodeposition cycles, the number of electrodeposition cycles being set such that each nanoparticle of the plurality of nanoparticles has a size at least an order of magnitude smaller than a lateral dimension of each nanowire of the array of nanowires.

In connection with any one of the aforementioned aspects, the electrodes, systems, and/or methods described herein may alternatively or additionally include or involve any combination of one or more of the following aspects or features. The substrate includes a semiconductor material. The semiconductor material is configured to generate charge carriers upon absorption of solar radiation such that the chemical cell is configured as a photoelectrochemical system. Each conductive projection of the array of conductive projections includes a nanowire configured to extract the charge carriers generated in the substrate. The substrate includes silicon. The semiconductor composition includes gallium nitride. The metallic composition includes tin. The metallic composition includes a metal oxide. Both ionic-like and covalent-like bonds are present at an interface between each nanoparticle of the plurality of nanoparticles and a respective conductive projection of the array of conductive projections. The size of each nanoparticle of the plurality of nanoparticles falls in a range from about 2 nanometers to about 3 nanometers. The lateral dimension of each conductive projection of the array of conductive projections falls in a range from about 30 nanometers to about 40 nanometers. The chemical cell is a thermochemical cell. An electrochemical system including a working electrode configured in accordance with one of the electrodes described herein, and further including a counter electrode, an electrolyte in which the working and counter electrodes are immersed, and a voltage source that applies a bias voltage between the working and counter electrodes. The bias voltage is set to a level for conversion of CO2 into formic acid at the working electrode. Both ionic-like and covalent-like bonds are present at an interface between each nanoparticle of the plurality of nanoparticles and a respective nanowire of the plurality of nanowires. The size of each nanoparticle of the plurality of nanoparticles falls in a range from about 2 nanometers to about 3 nanometers, and the lateral dimension of each nanowire of the array of nanowires falls in a range from about 30 nanometers to about 40 nanometers. A photoelectrochemical system including a working photocathode configured in accordance with one of the photocathode described herein, and further including a counter electrode, an electrolyte in which the working photocathode and the counter electrode are immersed, and a voltage source that applies a bias voltage between the working photocathode and the counter electrode. The bias voltage is set to a level for conversion of CO2 into formic acid at the working photocathode. Each nanoparticle of the plurality of nanoparticles includes tin oxide. The number of electrodeposition cycles falls in a range from about 60 cycles to about 80 cycles.

### BRIEF DESCRIPTION OF THE DRAWING FIGURES

For a more complete understanding of the disclosure, reference should be made to the following detailed description and accompanying drawing figures, in which like reference numerals identify like elements in the figures.

FIG. 1 is a schematic view and block diagram of an electrochemical system having a working electrode with a nanowire-nanoparticle architecture for catalytic conversion of carbon dioxide (CO₂) in accordance with one example.

FIG. 2 is a schematic, partial view of a photocathode having a nanowire array and nanoparticles for catalytic conversion of CO₂ in accordance with one example.

FIG. 3 is an energy band diagram of catalytic conversion of carbon dioxide (CO₂) into formic acid using the photocathode of FIG. 2.

FIG. 4 is a flow diagram of a method of fabricating an electrode with a nanowire array and nanoparticles for catalytic conversion of CO₂ in accordance with one example.

FIG. 5 depicts scanning electron microscopy (SEM) images of an array of nanowires before and after deposition of tin (Sn) nanoparticles in accordance with one example.

FIG. 6 depicts a number of plots comparing the efficiency, productivity, and other operational parameters of catalytic conversion of CO₂ using various photocathode architectures, including an architecture having a Gallium nitride (GaN) nanowire array with Sn nanoparticles in accordance with one example.

FIG. 7 depicts several SEM images of nanowires with nanoparticles deposited thereon, along with overlays of plots of nanoparticle distribution as a function of nanoparticle size, in accordance with a number of examples.

FIG. 8 is a graph depicting Faradaic efficiency for several examples of nanowire-nanoparticle architectures as a function of the number of nanoparticle deposition cycles.

FIG. 9 is a graph depicting turnover number (TON) for several examples of nanowire-nanoparticle architectures as a function of applied voltage.

The embodiments of the disclosed electrodes, systems, and methods may assume various forms. Specific embodiments are illustrated in the drawing and hereafter described with the understanding that the disclosure is intended to be illustrative. The disclosure is not intended to limit the invention to the specific embodiments described and illustrated herein.

### DETAILED DESCRIPTION OF THE DISCLOSURE

Electrodes of photoelectrochemical and other chemical cells having a conductive projection (e.g., nanowire) array with nanoparticles for reduction of carbon dioxide (CO₂) into formic acid are described. Methods of fabricating photocathodes and other electrodes for use in photoelectrochemical and other chemical systems are also described. The conductive projection (e.g., nanowire) array and the nanoparticles have semiconductor and metallic compositions, respectively, for catalytic conversion of carbon dioxide (CO₂) in the chemical cell. The compositions of the conductive projections (e.g., nanowires) and nanoparticles together provide a unique catalyst interface for CO₂ reduction. In some cases, the nanoparticles are sized at least an order of magnitude smaller than a lateral dimension of the conductive projection (e.g., nanowire) on which the nanoparticles are disposed.

Carbon dioxide activation relies on the intrinsic electronic properties of the electrocatalytic metals, and also depends on the catalytic architecture formed by these metals and their supports. For example, tin-based electrocatalysts are known to be intrinsically active for catalyzing CO₂ towards formic acid. However, the performance of tin (Sn) alone as an electrocatalyst is limited due to the lack of an efficient catalytic architecture. The disclosed methods and systems instead integrate Sn or other metallic compositions with a semiconductor support to develop an efficient architecture with superior interface catalytic properties for photoelectrocatalytic and other CO₂ reduction.

The electrocatalytic metal is supported by an architecture including a conductive projection (e.g., nanowire) array. One-dimensional (1-D) nanostructured metal nitrides, such as Gallium nitride (GaN) nanowires (GaN nanowires), are useful in solar fuels production and capable of being grown via molecular beam epitaxy (MBE) defect-free on planar silicon. The heterostructure of GaN nanowires presents a large surface-to-volume ratio, which is beneficial for sunlight harvesting and catalyst loading with a dramatically reduced amount, but high-density, of catalytic centers. Furthermore, the defect-free structure and high charge carrier mobility of GaN nanowires lead to charge carrier extraction from the silicon substrate. The unique electronic properties of gallium nitride are useful for activating the stable carbon dioxide molecule, thereby presenting a useful platform for supporting Sn and other nanoparticles to construct an ideal nanoarchitecture for solar-driven CO₂ conversion.

In some cases, the nanowires (e.g., GaN nanowires) are disposed on a planar semiconductor substrate (e.g., silicon) to provide a useful scaffold for loading Sn or other nanoparticles to construct a productive architecture (e.g., nanoarchitecture) for CO₂ conversion. The disclosed architectures may, in some cases, be free of noble metals. Nonetheless, high-efficiency sunlight collection is achieved via high-density active sites with a superior nanoparticle (e.g., Sn nanoparticle) atom-utilization efficiency, as well as effective charge carrier extraction.

The interface between the nanowires and nanoparticles presents covalent and ionic-like bonds for CO₂ activation. The ionic bonds indicate that the electron density is not equally distributed between the two atoms involved. For example, GaN:Sn nanoarchitectures exhibit an outstanding synergy for CO₂ activation through covalent Ga—C bonding and ionic-like Sn—O bonding. The interface presents a useful mechanism that is distinctly different from other CO₂ reduction involving, for instance, Sn-based electrodes. For instance, a turnover frequency (TOF) of 107 min⁻¹ for formic acid synthesis has been achieved with a current density of 17.5 mA cm⁻² and 76.9% Faradaic efficiency at −0.53 V under standard one-sun illumination, corresponding to an excellent productivity of 201 μmol·cm⁻²·h⁻¹, which is nearly two orders of magnitude higher than that of an electrode using a combination of only Sn and silicon. A stable operation time of 10 hours with a benchmarking turnover number of 64,000 has also been achieved. The disclosed electrodes thus support artificial photosynthesis of a value-added chemical from CO₂ using an architecture involving a substrate, conductive projections (e.g., nanowires) and nanoparticles having compositions of industry-ready materials (e.g., Si and GaN), and the earth-abundant catalyst (e.g., Sn).

Although described herein in connection with electrodes having GaN-based nanowire arrays for PEC CO₂ reduction, the disclosed electrodes are not limited to PEC reduction or GaN-based or other nanowires. A wide variety of types of chemical cells may benefit from use of the conductive projection (e.g., nanowire)-nanoparticle interface, including, for instance, electrochemical cells and thermochemical cells. Moreover, the nature, construction, configuration, characteristics, shape, and other aspects of the conductive projections, as well as the structures on or to which the conductive projections (e.g., nanowires) and/or nanoparticles are deposited, may vary. The disclosed electrodes, systems, and methods may also be directed to CO₂ reduction products other than or in addition to formic acid, such as CO, CH₃OH, CH₄, C₂H₄, C₂H₅OH, and C₂H₆.

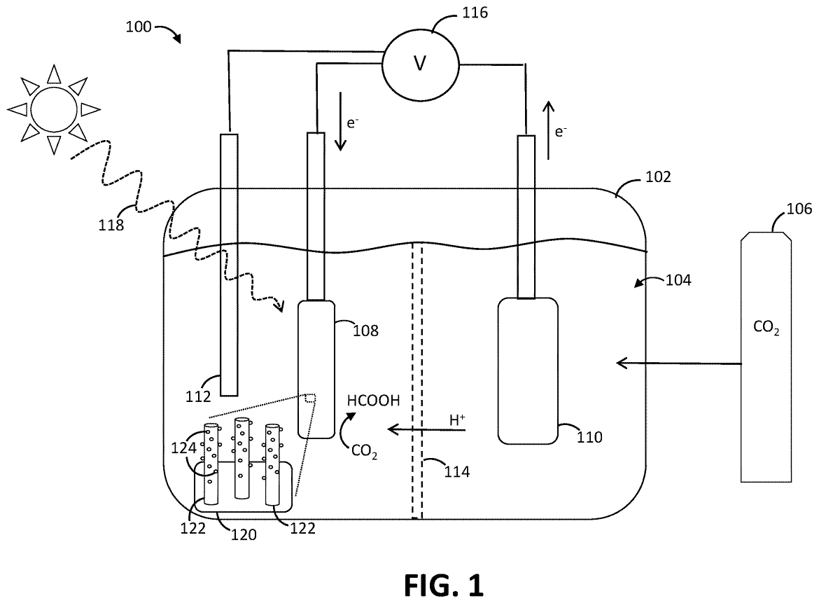

FIG. 1 depicts a system 100 for reduction of CO₂ into formic acid. The system 100 may also be configured for evolution of H₂. The system 100 may be configured as an electrochemical system. In this example, the electrochemical system 100 is a photoelectrochemical (PEC) system in which solar or other radiation is used to facilitate the CO₂ reduction. The manner in which the PEC system 100 is illuminated may vary. In thermochemical examples, the source of radiation may be replaced by a heat source.

The electrochemical system 100 includes one or more electrochemical cells 102. A single electrochemical cell 102 is shown for ease in illustration and description. The electrochemical cell 102 and other components of the electrochemical system 100 are depicted schematically in FIG. 1 also for ease in illustration. The cell 102 contains an electrolyte solution 104 to which a source 106 of CO₂ is applied. In some cases, the electrolyte solution is saturated with CO₂. Potassium bicarbonate KHCO₃ may be used as an electrolyte. Additional or alternative electrolytes may be used. Further details regarding an example of the electrochemical system 100 are provided below.

The electrochemical cell 102 includes a working electrode 108, a counter electrode 110, and a reference electrode 112, each of which is immersed in the electrolyte 104. The counter electrode 110 may be or include a metal wire, such as a platinum wire. The reference electrode 112 may be configured as a reversible hydrogen electrode (RHE). The configuration of the counter and reference electrodes 110, 112 may vary. For example, the counter electrode 110 may be configured as, or otherwise include, a photoanode at which water oxidation (2H₂O [symbol] O2+4e⁻+4H⁺) occurs.

Both reduction of CO₂ and evolution of H₂ may occur at the working electrode 112 as follows:

CO₂ reduction: CO₂+2H⁺+2e⁻ [symbol] CHOOH

H₂ evolution: 2H⁺+2e⁻ [symbol] H₂

To that end, electrons flow from the counter electrode 110 through a circuit path external to the electrochemical cell 102 to reach the working electrode 108. The working and counter electrodes 108, 110 may thus be considered a cathode and an anode, respectively. The competition between reduction of CO₂ and evolution of H₂ may be managed or controlled (e.g., to favor CO₂ reduction) via the composition of the components of the nanoarchitecture and/or the applied voltage, as described herein.

In the example of FIG. 1, the working and counter electrodes are separated from one another by a membrane 114, e.g., a proton-exchange membrane. The construction, composition, configuration and other characteristics of the membrane 114 may vary.

In this example, the circuit path includes a voltage source 116 of the electrochemical system 100. The voltage source 116 is configured to apply a bias voltage between the working and counter electrodes 108, 110. The bias voltage may be used to establish a ratio of CO₂ reduction to hydrogen (H₂) evolution at the working electrode, as described further below. The circuit path may include additional or alternative components. For example, the circuit path may include a potentiometer in some cases.

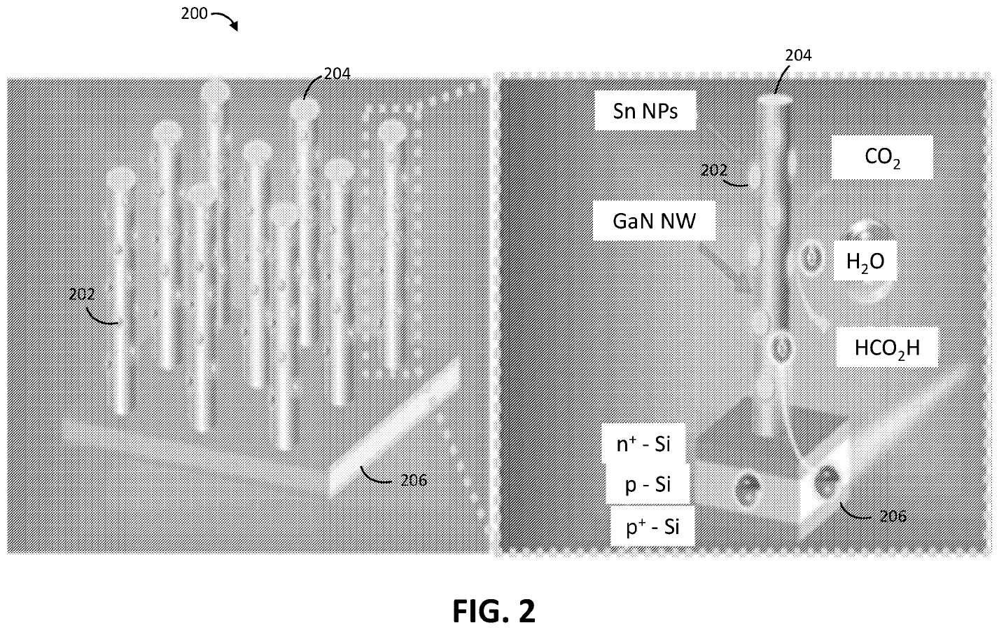

In some cases, the working electrode 108 is configured as a photocathode. Light 118, such as solar radiation, may be incident upon the working electrode 108 as shown. The electrochemical cell 102 may thus be considered and configured as a photoelectrochemical cell. In such cases, illumination of the working electrode 108 may cause charge carriers to be generated in the working electrode 108. Electrons that reach the surface of the working electrode 108 may then be used in the CO₂ reduction and/or the H₂ evolution. The photogenerated electrons augment the electrons provided via the current path. The photogenerated holes may move to the counter electrode for the water oxidation. A number of examples of, and further details regarding, photocathodes are provided below in connection with, for instance, FIGS. 2-4.

The working electrode 108 includes a substrate 120. The substrate 120 of the working electrode 108 may constitute a part of an architecture, or a support structure, of the working electrode 108. The substrate 120 may be uniform or composite. For example, the substrate 120 may include any number of layers or other components. The substrate 120 thus may or may not be monolithic. The shape of the substrate 120 may also vary. For instance, the substrate 120 may or may not be planar or flat.

The substrate 120 of the working electrode 108 may be active (functional) and/or passive (e.g., structural). In the latter case, the substrate 120 may be configured and act solely as a support structure for a catalyst arrangement formed along an exterior surface of the working electrode 108, as described below. Alternatively or additionally, the substrate 120 may be composed of, or otherwise include, a material suitable for the growth or other deposition of the catalyst arrangement of the working electrode 108.

The substrate 120 may include a light absorbing material. The light absorbing material is configured to generate charge carriers upon solar or other illumination. The light absorbing material has a bandgap such that incident light generates charge carriers (electron-hole pairs) within the substrate. Some or all of the substrate 120 may be configured for photogeneration of electron-hole pairs. To that end, the substrate 120 may include a semiconductor material. In some cases, the substrate 120 is composed of, or otherwise includes, silicon. For instance, the substrate 120 may be provided as a silicon wafer. The silicon may be doped. In some cases, the substrate 120 is heavily n-type doped, and moderately or lightly p-type doped. The doping arrangement may vary. For example, one or more components of the substrate 120 may be non-doped (intrinsic), or effectively non-doped. The substrate 120 may include alternative or additional layers, including, for instance, support or other structural layers. In other cases, the substrate 120 is not light absorbing. In these and other cases, one or more other components of the photocathode may be configured to act as a light absorber. Thus, in photoelectrochemical cases, the semiconductor material may be configured to generate charge carriers upon absorption of solar (or other) radiation, such that the chemical cell is configured as a photoelectrochemical system.

The substrate 120 of the working electrode 108 establishes a surface at which a catalyst arrangement of the electrode 108 is provided. The catalyst arrangement includes a conductive projection (e.g., nanowire)-nanoparticle architecture as described below.

The electrode 108 includes an array of nanowires 122 and/or other conductive projections supported by the substrate 120. Each nanowire 122 extends outward from the surface of the substrate 120. The nanowires 122 may thus be oriented in parallel with one another. Each nanowire 122 has a semiconductor composition for catalytic conversion of carbon dioxide (CO₂) in the chemical cell 102 into, e.g., formic acid. In some cases, the semiconductor composition includes Gallium nitride. Additional or alternative semiconductor materials may be used, including, for instance, indium nitride, indium gallium nitride, aluminum nitride, boron nitride, aluminum oxide, silicon, and/or their alloys.

The nanowires 122 may facilitate the conversion in one or more ways. For instance, each nanowire 122 may be configured to extract the charge carriers (e.g., electrons) generated in the substrate 120. The extraction brings the electrons to external sites along the nanowires 122 for use in the CO₂ reduction. The composition of the nanowires 122 may also form an interface well-suited for reduction of CO₂, as explained below.

Each nanowire 122 may be or include a columnar, post-shaped, or other elongated structure that extends outward (e.g., upward) from the plane of the substrate 120. The nanowires 122 may be grown or formed as described in U.S. Pat. No. 8,563,395, the entire disclosure of which is hereby incorporated by reference. The dimensions, size, shape, composition, and other characteristics of the nanowires 122 (and/or other conductive projections) may vary. For instance, each nanowire 122 may or may not be elongated like a nanowire. Thus, other types and shapes of conductive projections from the substrate 120, such as various shaped nanocrystals, may be used.

In some cases, one or more of the nanowires 122 is configured to generate electron-hole pairs upon illumination. For instance, the nanowires 122 may be configured to absorb light at frequencies different than other light absorbing components of the electrode 108. For example, one light absorbing component, such as the substrate 120, may be configured for absorption in the visible or infrared wavelength ranges, while another component may be configured to absorb light at ultraviolet wavelengths. In other cases, the nanowires 122 are the only light absorbing component of the electrode 108.

The electrode 108 further includes nanoparticles 124 disposed over the array of nanowires 122. Each nanoparticle 124 has a metallic composition for the catalytic conversion of carbon dioxide (CO₂) in the chemical cell 102. A plurality of the nanoparticles 124 are disposed on each nanowire 122, as schematically shown in FIG. 1. The nanoparticles 124 are distributed across the outer surface of each nanowire 122. For example, each nanowire 122 has a plurality of the nanoparticles 124 distributed across or along sidewalls of the nanowire 122. The distribution may not be uniform or symmetric as shown. As described herein, each nanoparticle 124 may include or be composed of a metal catalyst for reduction of carbon dioxide (CO₂) in a photoelectrochemical cell.

The metallic composition may be or include a pure (e.g., elemental) metal composition and/or a pure metal oxide composition and/or a composition involving metal alloys. In some cases, the metallic composition of the nanoparticles 124 includes tin (Sn). Sn-based nanoparticles are configured for the conversion of CO₂ into formic acid, as described herein. Alternative or additional metal catalysts may be used, including, for instance, copper (Cu), lead (Pb), and indium (In). The use of alternative or additional metals and/or metal oxides in the metallic composition may lead to alternative or additional reduction products of the CO₂ conversion. In some cases, one or more noble metals, such as gold, may be added to the metallic composition.

The metallic composition may alternatively or additionally include a metal oxide of the metal. Thus, each nanoparticle 124 may also include Tin oxide. For instance, each nanoparticle 124 may include a Sn core surrounded by an outer layer of Tin oxide (SnOₓ). The arrangement of the metal and metal oxide may vary, including, for instance, in connection with the environment in, and procedure by, which the nanoparticles 124 are deposited or formed.

The metallic composition of the nanoparticles 124 may or may not include an elemental or purified metal. Alternatively, a metal alloy or other metal-based material may be used.

The nanoparticles 124 may be sized in a manner to facilitate the CO₂ reduction. The size of the nanoparticles 124 may be useful in catalyzing the reaction, as described herein. The size of the nanoparticles 124 may be promote the CO₂ reduction in additional or alternative ways. For instance, the nanoparticles 124 may also be sized to avoid inhibiting the illumination of the light absorber (e.g. the substrate 120).

In some cases, each nanoparticle 124 has a size at least an order of magnitude smaller than a lateral dimension (e.g., a diameter) of each nanowire 122. For example, the size of each nanoparticle 124 may fall in a range from about 2 nanometers (nm) to about 3 nm, while the lateral dimension of each nanowire 122 may fall in a range from about 30 nm to about 40 nm, although other sizes and dimensions may be used.

The combination of the nanowires 122 and the nanoparticles 124 may promote the CO₂ reduction in other ways. For instance, the respective compositions of the nanowires 122 and the nanoparticles 124 may result in a co-catalytic interface having bonds well-suited for the CO₂ reduction. In some cases, both ionic-like and covalent-like bonds are present at the interface between each nanoparticle 124 and a respective nanowire 122.

The manner in, or extent to, which the array of nanowires 122 is ordered may vary. In some cases, the nanowires 122 may be arranged laterally in a regular or semi-regular pattern. In other cases, the lateral arrangement of the nanowires 122 is irregular. In such cases, the ordered nature of the nanowires 122 is instead limited to the parallel orientation of the nanowires 122.

The distribution of the nanoparticles 124 may be uniform or non-uniform. The nanoparticles 124 may thus be distributed randomly across each nanowire 122. The schematic arrangement of FIG. 1 is shown for ease in illustration.

The nanowires 122 and the nanoparticles 124 are not shown to scale in the schematic depiction of FIG. 1. The shape of the nanowires 122 and the nanoparticles 124 may also vary from the example shown. Further details regarding Sn-based example nanoparticles and GaN-example nanowires are provided below.

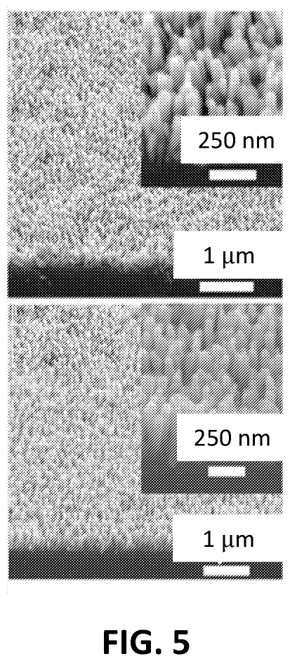

A photocathode having Sn nanoparticles and GaN nanowires was fabricated on a Si substrate via nanostructure-engineering. In one example, molecular beam epitaxial (MBE) growth of GaN nanowires on n⁺-p silicon junction was followed by electrodeposition of Sn nanoparticles. Electrodeposition of the nanoparticles may be configured to realize a desired size. Smaller nanoparticles may be achieved with electrodeposition relative to other deposition procedures. Electrodeposition may also be used with other metallic compositions for the nanoparticles. Further details regarding example fabrication procedures are provided below, e.g., in connection with FIG. 5.

FIG. 2 depicts an example architecture 200 having Sn nanoparticles 202 and GaN nanowires 204 on a Si substrate 206. The Si substrate 206, an earth-abundant material, is doped or otherwise formed to include n+, p, and p+ layers as shown. In this case, the layers are arranged with the n+ layer adjacent or otherwise closest to the nanowires 204, and the p layer between the n+ and p+ layers. The layers present an n⁺-p silicon junction for the growth of the nanowires 204. The substrate 206 may thus provide a narrow bandgap (about 1.1 eV) that is readily photoexcited by a large part of the solar spectrum to generate electron-hole pairs for the reaction. The light absorption of the GaN nanowires 204 may be neglected because of the large bandgap of GaN (about 3.4 eV). However, the GaN nanowires 204 may improve the optical and electronic properties between planar silicon and Sn-based cocatalysts due to the unique geometry of the nanowires 204 and a strong charge carrier extraction effect. The GaN nanowires 204 may also function as an excellent geometric and catalytic modifier to load the Sn-based cocatalyst for accelerating the reaction.

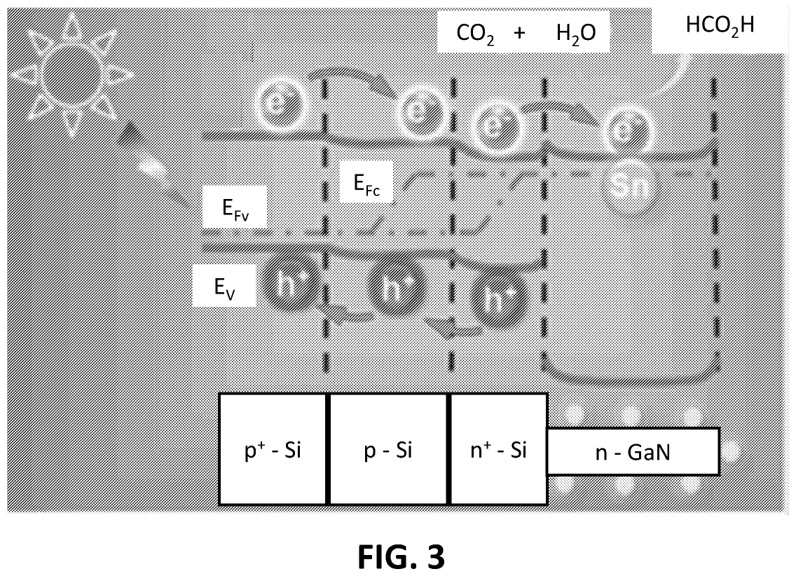

The architecture 200 is configured to provide light harvesting, charge carrier extraction, and catalytic functions that are spatially decoupled. As a result, the optical, electronic, and catalytic properties can be rationally tuned to achieve superior performance. The corresponding energy diagram of the electrode is shown in FIG. 3. In that example, both the GaN nanowires and the silicon substrate are heavily n-type doped. The electron-migration energy barrier between them is thus negligible.

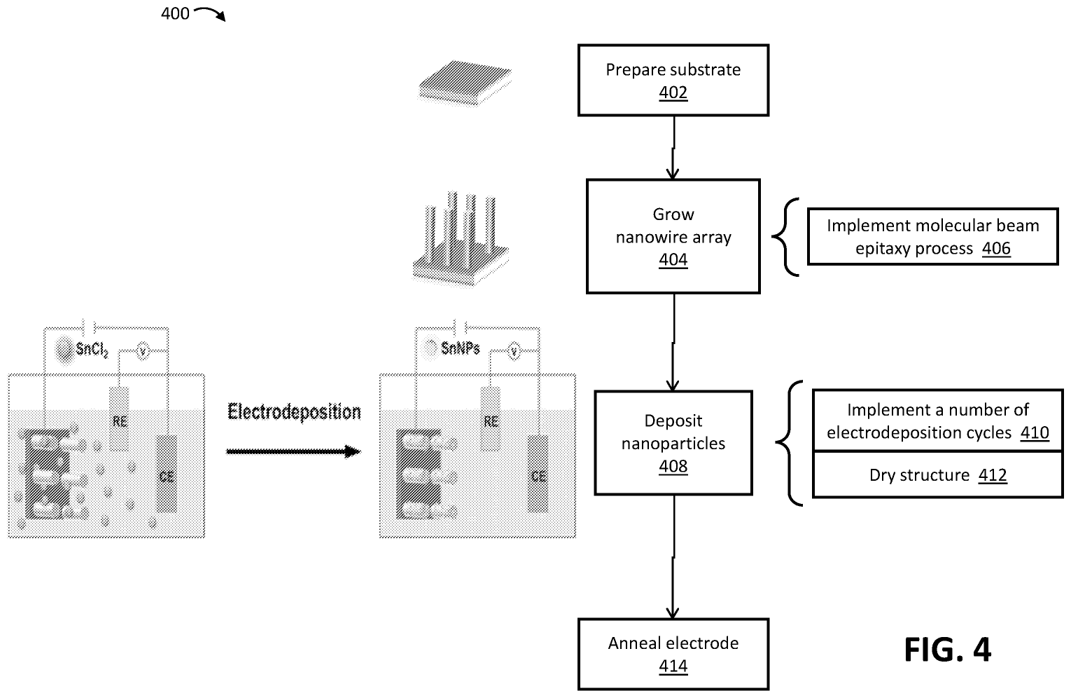

FIG. 4 depicts a method 400 of fabricating an electrode of an electrochemical system in accordance with one example. The method 400 may be used to manufacture any of the working electrodes described herein or another electrode. The method 400 may include additional, fewer, or alternative acts. For instance, the method 400 may or may not include one or more acts directed to growing a nanowire array (act 404).

The method 400 may begin with an act 402 in which a substrate is prepared. The substrate may be or be formed from a p-n Si wafer. In one example, a 2-inch Si wafer was used, but other (e.g., larger) size wafers may be used. Other semiconductors and substrates may be used. Preparation of the substrate may include one or more thermal diffusion procedures.

In some cases, an n+-p silicon junction of the substrate is formed through a standard thermal diffusion process using, e.g., a (100) silicon wafer. Phosphorus and boron as n-type and p-type dopants, respectively, were deposited on the front and back sides of the polished p-Si (100) wafer by spin-coating, but other dopants may be used. The wafer may then be annealed, e.g., at 900 C under argon atmosphere for four hours.

In the example of FIG. 4, the method 400 includes an act 404 in which GaN or other nanowire arrays (or other conductive projections) are grown or otherwise formed on the substrate. Each nanowire (or other conductive projection) has a semiconductor composition for catalytic conversion of carbon dioxide (CO₂), as described herein. The nanowire growth may be achieved in an act 406 in which plasma-assisted molecular beam epitaxy (MBE) is implemented. The act 406 may be implemented under nitrogen-rich conditions. In one example, the growth conditions were as follows: a growth temperature of 790° C. for 1.5 hours, a Ga beam equivalent pressure of about 6×10⁻⁸ Torr, a nitrogen flow rate of 1 standard cubic centimeter per minute (sccm), and a plasma power of 350 Watts. The substrate and the nanowires provide or act as scaffolding for the catalysts deposited in the following steps.

In an act 408, nanoparticles are deposited across each nanowire (or other conductive projection). Each nanoparticle has a metallic composition for the catalytic conversion of carbon dioxide (CO₂), as described herein. The nanoparticles are deposited across one or more outer surfaces of the nanowires. Each nanoparticle may be composed of, or otherwise include, a metal, as described herein. The act 408 may include implementation of a number of cycles of an electrodeposition process in an act 410, after which the structure is dried in an act 412. Alternative or additional deposition procedures may be used. Further details regarding examples of the particle deposition are provided below.

The electrodeposition process may include cyclic voltammetry. In one example, the GaN nanowire and Si substrate scaffolding was immersed into a SnCl₂ aqueous solution (e.g., 200 mL×1 mmol L⁻¹). The electrodeposition was carried out in a PEC chamber by a typical three-electrode configuration (a schematic view of an example of which is shown in FIG. 4), employing Ag/AgCl as a reference electrode and Pt as a counter electrode. The first depositing step was realized by sweeping potential between +0.1 to +2.0 V, followed by another sweeping deposition at the potential range of −0.5 V to −2.0 V, with a desired number of cycles. The scanning rate may be 100 mV/s. The synthesized sample may be thoroughly washed with distilled water after the deposition.

The loading amount and the size of the Sn nanoparticles may be tailored by tuning the depositing cycle number. For instance, the number of electrodeposition cycles may be set such that each nanoparticle of the plurality of nanoparticles has a size at least an order of magnitude smaller than a lateral dimension of each nanowire of the array of nanowires. In some cases, the number of electrodeposition cycles falls in a range from about 60 cycles to about 80 cycles, but the range may vary based on other parameters or factors, including, for instance, the type of catalysts. In one example, about 70 electrodeposition cycles were implemented.

In some cases, the method 400 includes an act 414 in which the electrode is annealed. One example electrode was annealed at 400° C. for 10 minutes in forming gas (e.g., 5% H₂, balance N₂) at a flow rate of 200 sccm. The parameters of the anneal process may vary.

Details regarding photoelectrochemical (PEC) performance of examples of the nanowire-nanoparticle architectures of the disclosed PEC electrodes are now provided in connection with FIGS. 5-9.

FIG. 5 depicts scanning electron microscopy (SEM) characterization of an example GaN—Sn architecture. The SEM characterization shows that the GaN nanowires are vertically aligned on the planar silicon substrate. In this example, the length of the GaN nanowires is about 300 nm with a diameter of about 40 nm. After electrodeposition, the overall morphology of the nanowire arrays was not affected. Based on UV-visible reflectance spectral measurements, the GaN nanowires were shown to indeed perform as an effective antireflection coating for improving the sunlight harvesting of the silicon substrate in a wide wavelength range of about 200 to about 1100 nm. The Sn nanoparticle/GaN nanowire/Si architecture exhibits a further improvement in light absorption compared to bare GaN nanowires on a Si substrate, thus enhancing the photocurrent of the chemical cell. Energy dispersive X-ray spectroscopy (EDX) analysis also confirmed that Sn was successfully deposited on the GaN nanowires/Si scaffolds. A particle-like contrast was observed for Sn nanoparticle-decorated GaN nanowires in a high angle annular dark-field scanning transmission electron microscopy (STEM-HAADF) image, whose features with brighter intensities suggest the presence of Sn nanoparticles, insofar as HAADF imaging provides Z-sensitive contrast, where Z is the effective atomic number. In this example, the Sn nanoparticles were uniformly dispersed on the GaN nanowires and had diameters of about 2.3 nm. Using inductively coupled plasma-atomic emission spectroscopy (ICP-AES), the loading density of Sn was determined to be only 0.031 μmol cm⁻² (normalized to the geometric surface area). These results confirm the distinct catalyst-utilization efficiency with high-density catalytic centers.

Electron energy loss spectroscopy (EELS) mapping further revealed that, at the positions corresponding to particle-like features in the HAADF image, Sn and oxygen (O) are coexisted, indicating that Sn nanoparticles are likely a mixture of Sn and SnOₓ. In high-resolution TEM imaging, lattice fringes were clearly observed with an average lattice spacing of ca. 0.335 nm, which is attributed to the (110) interplanar spacing of SnO₂. The X-ray photoelectron spectroscopy (XPS) depth profile of high-resolution Sn 3d spectral demonstrated that the peaks at 493.4 eV (Sn 3d3/2) and 484.9 eV (Sn 3d5/2) assigning to Sn increase with the etching time, while the features of SnOₓ at 495.6 eV (Sn 3d3/2) and 487.2 eV (Sn 3d5/2) reduce concurrently. This further confirms that Sn nanoparticles included Sn and SnOₓ, with the Sn being covered with SnOₓ. XPS measurement indicated that both Co nanoparticles and Ni nanoparticles were covered with metal oxides as well. The SnOₓ in the Sn/SnOₓ mixture may be useful for HCOOH synthesis.

The featured peaks of Ga 3d and N 1s of the Sn:GaN nanoarchitecture exhibit an obvious shift compared to that of bare GaN. That suggests a redistribution of the electron density between GaN and Sn nanoparticles. The redistribution of the electron density indicates a strong interaction between GaN and Sn nanoparticles. In the catalytic cycle, this redistribution is useful for the activation of CO₂ and hence tailors the catalytic properties for PEC CO₂ reduction reactions. HAADF imaging depicted that the lattice space between the two adjacent (002) plane is 0.26 nm, indicating the growth direction of GaN nanowires along the c-axis. The as-grown GaN is defect-free and the edge of GaN nanowire is atomically sharp and flat. This aspect of the nanowires is useful for charge carrier extraction. Together, these results indicate that the GaN:Sn nanoarchitecture on silicon platform is useful for CO₂ reduction.

Linear sweep voltammetry (LSV) measurements of bare silicon, GaN nanowires on a Su substrate, and GaN nanowires on Si decorated with various cocatalysts were conducted in CO₂-purged 0.1 M aqueous solution of KHCO₃ under standard one-sun illumination. All of the reactions described herein were carried out under this condition, unless indicated otherwise. Pt wire and Ag/AgCl were used as counter electrode and reference electrode, respectively.

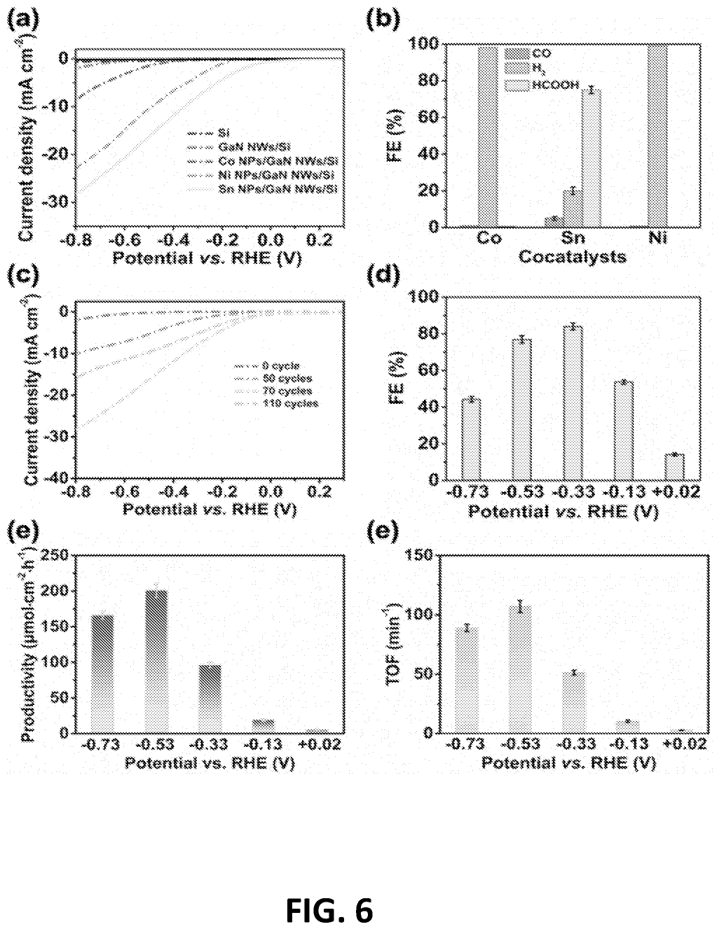

As shown in FIG. 6, although silicon has a suitable bandgap for absorbing the large fraction of sunlight, the photocurrent of bare silicon is almost not observed in the examined potential range from +0.3 to −0.8 V due to the fast surface recombination of electrons and holes. Both the onset potential and current density of bare silicon are improved by incorporating GaN nanowires, attributing to the enhanced sunlight collection, effective charge carrier extraction, and reduced surface recombination. However, the onset potential of GaN nanowires/Si with a current density of −0.2 mA cm-2 is still highly negative at −0.5 V as a result of slow electron kinetics without cocatalysts.

With continued reference to FIG. 6, Co-, Ni-, and Sn-based cocatalysts were respectively introduced to the GaN nanowire/Si scaffolds by the same electrodepositing procedure. These earth abundant cocatalysts were chosen because of their previously reported relatively high activity for CO₂ reduction. The J-V curve of the GaN nanowire/Si scaffolds exhibits a substantial enhancement after incorporating Co-, Ni-, and Sn-based cocatalysts. The effect of Sn nanoparticles is more pronounced than that of Co and Ni. A favorable onset potential of +0.22 V is realized using Sn nanoparticles in combination with GaN nanowires and a Si substrate. The photocurrent density can reach −28.2 mA cm⁻² at −0.8 V. The improvement can be ascribed to the deposited Sn nanoparticles enhancing the electron-hole separation and offering active sites for boosting the catalytic activity. Moreover, the decoration of metallic Sn core can reduce the upward band bending of n-type doped GaN because of the neighboring inhomogeneous Schottky's barrier, thereby reducing the voltage loss of the device. Among the three cocatalysts, only the Sn nanoparticles are catalytically active for formic acid formation.

Nuclear magnetic resonance spectroscopy measurement showed that no other liquid products were produced and the Faradaic efficiency (FE) of formic acid was as high as 76.9% while Co- and Ni-based cocatalysts primarily produce hydrogen with only trace amounts of CO production (FE<1%) under the same conditions.

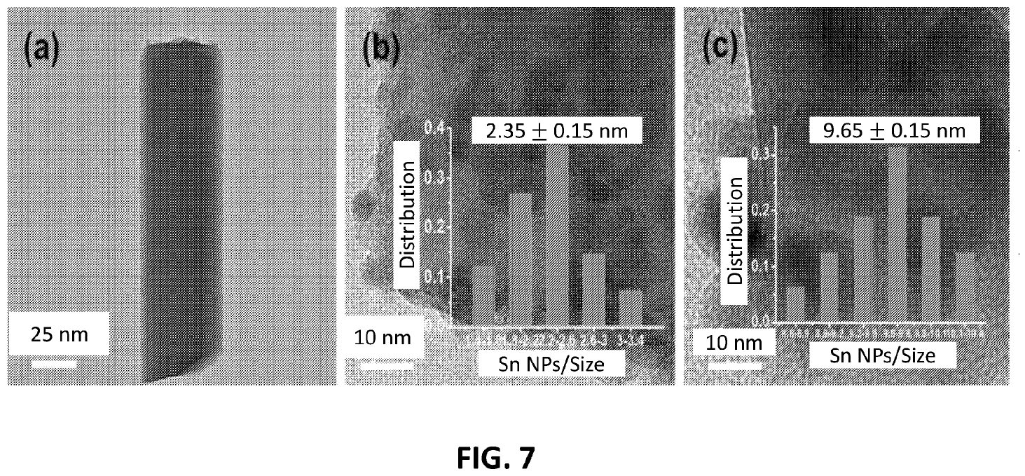

The performance of the device was further optimized by tuning the depositing cycle number of Sn nanoparticles, which determines the size and distribution of Sn nanoparticles. As illustrated in FIGS. 7-9, at an initial stage of 0 to 70 cycles, both the activity and Faradaic efficiency are enhanced by increasing the cycle number. A maximum FE of 76.9% with the highest total current density of 17.5 mA cm-2 was obtained at 70 cycles with Sn nanoparticle size of ca. 2.35 nm. However, at a higher loading of 110 cycles, the performance dramatically decreased as Sn nanoparticle size increased up to ca. 9.65 nm.

The underlying cause for this phenomenon is that the reaction is influenced by Sn nanoparticles in opposite ways. In the catalytic process, Sn nanoparticles offer active sites for enhancing electron-hole separation and catalyzing the reaction. Both the catalytic activity and selectivity were first improved because of the increasing number of active sites. For larger cycle number, however, overloading of Sn nanoparticles would shield the light absorption. Moreover, TEM images of FIG. 7 illustrate that the diameters of Sn nanoparticles increase significantly with the depositing cycle number, lowering its activity. These two factors lead to a reduced catalytic performance. Therefore, in some cases, about 70 cycles is useful for the electrodeposition, insofar as it achieves a suitable balance of sufficient catalytic sites, effective sunlight harvesting, and effectively sized Sn nanoparticles with high activity.

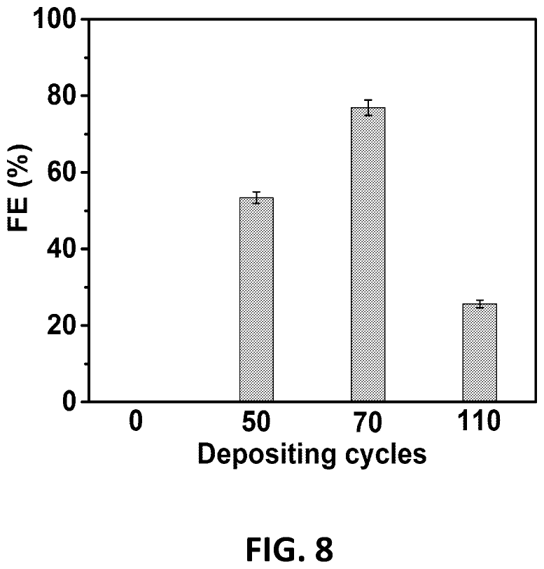

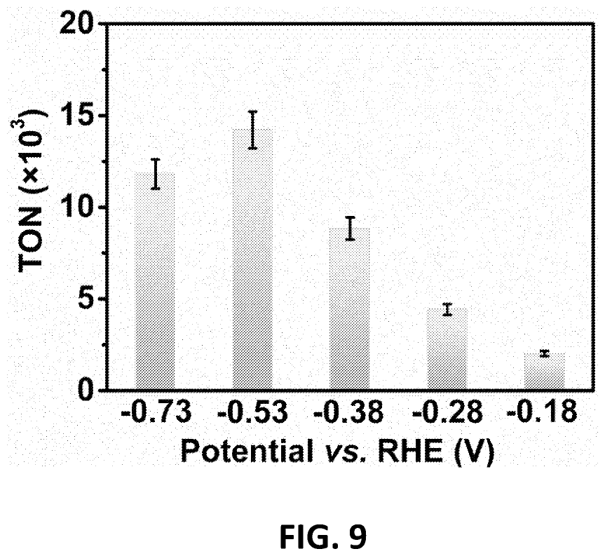

FIG. 7 depicts TEM images of Sn nanoparticles/GaN nanowires/Si with the following Sn nanoparticle depositing cycles: (i) 0 cycle, (ii) 70 cycles, and (iii) 110 cycles. FIG. 8 depicts FE for HCOOH production of Sn nanoparticles/GaN nanowires/Si with various depositing cycles of Sn nanoparticles in CO₂-purged 0.1 M KHCO₃ under standard one-sun illumination at −0.53 V vs. RHE. FIG. 9 depicts the influence of the applied potential on the turnover number of Sn nanoparticles/GaN nanowires/Si for formic acid formation in CO₂-purged 0.1 M KHCO₃ under standard one-sun illumination for 2 hours.

With reference again to FIG. 6, the influence of the applied potentials on Faradaic efficiency of formic acid was also investigated. The Faradaic efficiency exhibited a volcano-like trend as a function of the applied bias. It showed a maximum of 84% at −0.33 V. As the potential shifted negatively, HCOOH Faradaic efficiency, however, decreased with increasing current density. This can be explained by that at highly negative potentials, the pH of the electrolyte near the electrode is remarkably higher than that in the bulk electrolyte because of the release of OH⁻ from the reaction. It will decline the local CO₂ concentration of the cathode surface, thus leading to degeneration in Faradaic efficiency of formic acid. When the potential further shifts to −0.73V, HCOOH Faradaic efficiency decreases to 44.3% because of the severe hydrogen evolution, which competes with CO₂-towards-HCOOH conversion under these conditions. With positive potential shifting, HCOOH Faradaic efficiency also reduces. Impressively, formic acid, however, is produced with 14.2% Faradaic efficiency at underpotential of 220 mV (+0.02 V vs. RHE), where solar light is the only energy force for driving the reaction The equilibrium redox potential E\hat{ }O(CO₂/HCOOH)=−0.20 V vs. RHE. At more positive potential, the formation of formic acid is negligible. It indicates that +0.02 V is the onset potential for formic acid generation, which is more negative than that of hydrogen evolution at +0.22 V due to the difficulty of CO₂ activation compared to proton reduction. In addition, the reaction did not happen in dark or under illumination without external circuit, suggesting that the CO₂ reduction proceeded via photoelectrocatalysis.

To evaluate the activity of an architecture including Sn nanoparticles, GaN nanowires, and a Si substrate, the productivity for formic acid under different potentials was measured, as shown in FIG. 6. At +0.02 V, the productivity was 4.9 μmol·cm⁻²·h⁻¹. It increased with the negative shift of the potential and reached a maximum of 201 μmol·cm⁻²·h⁻¹ at −0.53 V. However, a slight reduction in productivity to 166 μmol·cm⁻²·h⁻¹ was found at −0.73 V as a consequence of the severe competition of hydrogen evolution. To determine the activity more accurately, the turnover frequency (TOF) and turnover number (TON) are also shown in FIG. 6. A favorable TOF of 2.6 min⁻¹ was obtained at the onset of +0.02 V, corresponding to a TON of 312. The TOF increased significantly with the increasing current density as the potential negatively shifted. Strikingly, a maximum TOF of 107 min⁻¹ for formic acid with a high TON of 12,800 was achieved at −0.53 V within two hours, which is much higher than that of state-of-the-art solar-driven CO₂-into-HCOOH conversion.

The superior TOF is primarily due to the prominent synergy of the GaN:Sn nanoarchitecture for CO₂ bond activation. In addition, one-dimensional GaN nanowire arrays also play a role in the outstanding performance by enhancing the sunlight absorption of the planar silicon wafer because of the anti-reflection effect and by promoting the superior catalyst-utilization efficiency. Moreover, electrochemical impedance spectroscopy (EIS) analysis suggests that the charge carrier transfer resistance of an architecture of Sn nanoparticles, GaN nanowires, and a Si substrate is over one order of magnitude smaller compared to that of Sn/Si, indicating that GaN nanowires function as an efficient electron-migration channel for charge carriers separation. Such a distinct effect is mainly due to the negligible conduction band offset between GaN and Si as well as the high electron mobility of defect-free GaN. Control experiments further revealed that, without the use of GaN nanowires, a Sn/Si planar structure shows much worse LSV behavior compared to an architecture of Sn nanoparticles, GaN nanowires, and a Si substrate. In addition, the productivity of an architecture of Sn nanoparticles, GaN nanowires, and a Si substrate was nearly two orders of magnitude higher than that of Sn nanoparticles/Si (2.1 μmol·cm⁻²·h⁻¹). These results indicate that GaN nanowires are useful for improving performance due to structural, optical, and electronic properties. For instance, without Sn nanoparticles, it is found that GaN nanowires on a Si substrate is not active for formic acid synthesis, confirming the cooperative effect between GaN nanowires and Sn nanoparticles for high-efficiency formic acid formation.

To further elucidate the synergy between GaN nanowires and Sn nanoparticles at the atomic level, density functional theory calculations were performed to study the interaction between Sn nanoparticles and GaN nanowires, CO₂ adsorption at the interface of Sn nanoparticles and GaN nanowires, and potential reaction pathways for reducing CO₂ to HCOOH. Based on the experimental result that Sn nanoparticles are featured by SnOₓ shell, Sn₁₃O₂₆/GaN(1010) is established to study the interfacial properties of Sn: GaN nanoarchitecture; and the hydroxylation of Sn nanoparticles was used in the calculations due to the effect of PEC CO₂ reduction conditions in an aqueous environment.

Strong electronic coupling between Sn nanoparticles and GaN nanowires was evidenced by the electron charge density redistribution around the interfacial region. Apparent electron reduction is found near the Ga atoms while electron accumulation occurs around the neighboring O atoms, indicating an ionic-like Ga—O bonding. Meanwhile, notable electron accumulation around the middle region of Sn and N atoms suggests the formation of covalent Sn—N bonding. The results indicate a strong interaction between Sn nanoparticles and GaN nanowires, i.e., ionic-like Ga—O bonding and covalent Sn—N bonding, altering the electronic properties of the interface, which is likely useful for the activation of CO₂.

Stability testing of the disclosed electrodes was also performed. The photocurrent density did not show observable degradation after 10 hours of irradiation. The HCOOH Faradaic efficiency was relatively stable with a mixture of CO and H₂, an important chemical feedstock named syngas, obtained as the main byproducts. No other gaseous products were detected by gas chromatography. The morphology of the nanowire arrays and the oxidation states of the elements of the device did not change before and after the reaction. These results confirm the stability of the disclosed architectures, as gallium nitride is capable of functioning as an efficient protection layer against corrosion as suggested by our previous study.

The TON for formic acid was as high as 64,000 with an outstanding TOF of 107 min⁻¹ during a relatively stable operation of 10 hours. Isotopic measurements were carried out to identify the carbon source of formic acid. 1H-NMR analysis revealed that under CO₂-purged HCO₃₋ electrolyte solution (0.1 M), a singlet peak of HCOOH (δ 8.35 ppm) was observed. When the reaction was performed under ¹³CO₂ atmosphere with H¹³CO₃⁻ as the electrolyte, a doublet peak is illustrated at 8.17 and 8.57 ppm, which is credited to the proton coupled to ¹³C in H¹³COOH. In contrast, when the reaction was carried out in argon-purged Na₂SO₄ aqueous solution, no signal of HCOOH was observed in 1H-NMR spectra. These results indicate that the carbon source of HCOOH originates from CO₂ (either from CO₂ purging or from bicarbonate dissociation) rather than from the impurities in the electrolyte and photocathode. Additionally, it should be noted that water is the only reductant for CO₂ conversion without any sacrificial agents, illustrating an authentic artificial photosynthetic route.

The disclosed nanoarchitectures (e.g., GaN:Sn nanoarchitectures) may be formed using a combination of molecular beam epitaxy and electrodeposition. The nanoarchitectures may be formed (e.g., directly) on a planar substrate (e.g., silicon) for artificial photosynthesis of formic acid from CO₂ using H₂O as the only reductant. Such a multifunctional architecture allows for efficient solar light harvesting, effective charge carrier extraction, and exposing active sites of Sn nanoparticles with superior atom-utilization efficiency. The GaN:Sn nanoarchitecture cooperates well for activating CO₂ through covalent Ga—C bonding and ionic-like Sn-O bonding at the interface, showing a useful and energetically-favorable mechanism for formic acid synthesis. Formic acid is produced at an underpotential of 220 mV (+0.02 V vs. RHE); and an astonishing TOF of 107 min⁻¹ is obtained at a low potential of −0.53 V with 17.5 mA·cm⁻² and 76.9% FE under standard one-sun illumination, corresponding to an appreciable productivity of 201 μmol·cm⁻²·h⁻¹. Stable operation (e.g., 10 hours) is also achieved with a high turnover number (e.g., 64,000). The disclosed electrodes (e.g., photocathodes) are composed of, or otherwise include, industry-ready materials, e.g., Si and GaN, and an earth-abundant, nontoxic catalyst (e.g., Sn). The disclosed electrodes may be manufactured using standard semiconductor processing. As such, the disclosed electrodes provide a promising route for achieving low-cost, high-efficiency, and robust artificial photosynthesis for the production for solar fuels and high-value chemicals from CO₂ conversion.

The present disclosure has been described with reference to specific examples that are intended to be illustrative only and not to be limiting of the disclosure. Changes, additions and/or deletions may be made to the examples without departing from the spirit and scope of the disclosure.

The foregoing description is given for clearness of understanding only, and no unnecessary limitations should be understood therefrom.

What is claimed is:

1. An electrode of a chemical cell, the electrode comprising:
a substrate having a surface;
an array of conductive projections supported by the substrate and extending outward from the surface of the substrate, each conductive projection of the array of conductive projections having a semiconductor composition for catalytic conversion of carbon dioxide (CO₂) in the chemical cell; and
a plurality of nanoparticles disposed over the array of conductive projections, each nanoparticle of the plurality of nanoparticles having a metallic composition for the catalytic conversion of carbon dioxide (CO₂) in the chemical cell;
wherein each nanoparticle of the plurality of nanoparticles has a size at least an order of magnitude smaller than a lateral dimension of each conductive projection of the array of conductive projections.

2. The electrode of claim 1, wherein:
the substrate comprises a semiconductor material; and
the semiconductor material is configured to generate charge carriers upon absorption of solar radiation such that the chemical cell is configured as a photoelectrochemical system.

3. The electrode of claim 2, wherein each conductive projection of the array of conductive projections comprises a nanowire configured to extract the charge carriers generated in the substrate.

4. The electrode of claim 1, wherein the substrate comprises silicon.

5. The electrode of claim 1, wherein the semiconductor composition comprises gallium nitride.

6. The electrode of claim 1, wherein the metallic composition comprises tin.

7. The electrode of claim 1, wherein the metallic composition comprises a metal oxide.

8. The electrode of claim 1, wherein both ionic-like and covalent-like bonds are present at an interface between each nanoparticle of the plurality of nanoparticles and a respective conductive projection of the array of conductive projections.

9. The electrode of claim 1, wherein the size of each nanoparticle of the plurality of nanoparticles falls in a range from about 2 nanometers to about 3 nanometers.

10. The electrode of claim 1, wherein the lateral dimension of each conductive projection of the array of conductive projections falls in a range from about 30 nanometers to about 40 nanometers.

11. The electrode of claim 1, wherein the chemical cell is a thermochemical cell.

12. An electrochemical system comprising a working electrode configured in accordance with the electrode of claim 1, and further comprising:
a counter electrode;
an electrolyte in which the working and counter electrodes are immersed; and
a voltage source that applies a bias voltage between the working and counter electrodes;
wherein the bias voltage is set to a level for conversion of CO₂ into formic acid at the working electrode.

13. A photocathode for a photoelectrochemical cell, the photocathode comprising:
a substrate comprising a light absorbing material, the light absorbing material being configured to generate charge carriers upon solar illumination;
an array of nanowires supported by the substrate, each nanowire of the array of nanowires being configured to extract the charge carriers from the substrate, each nanowire of the array of nanowires comprising gallium nitride; and
a plurality of nanoparticles distributed across each nanowire of the array of nanowires, each nanoparticle of the plurality of nanoparticles having a metallic composition for the catalytic conversion of carbon dioxide (CO₂) in the photoelectrochemical cell into formic acid;
wherein each nanoparticle of the plurality of nanoparticles has a size at least an order of magnitude smaller than a lateral dimension of each nanowire of the array of nanowires.

14. The photocathode of claim 13, wherein the substrate comprises silicon.

15. The photocathode of claim 13, wherein the metallic composition comprises tin.

16. The photocathode of claim 13, wherein the metallic composition comprises a tin oxide.

17. The photocathode of claim 13, wherein both ionic-like and covalent-like bonds are present at an interface between each nanoparticle of the plurality of nanoparticles and a respective nanowire of the plurality of nanowires.

18. The photocathode of claim 13, wherein:
the size of each nanoparticle of the plurality of nanoparticles falls in a range from about 2 nanometers to about 3 nanometers; and
the lateral dimension of each nanowire of the array of nanowires falls in a range from about 30 nanometers to about 40 nanometers.

19. A photoelectrochemical system comprising a working photocathode configured in accordance with the photocathode of claim 13, and further comprising:
a counter electrode;
an electrolyte in which the working photocathode and the counter electrode are immersed; and
a voltage source that applies a bias voltage between the working photocathode and the counter electrode;
wherein the bias voltage is set to a level for conversion of CO₂ into formic acid at the working photocathode.

20. A photocathode for a photoelectrochemical cell, the photocathode comprising:
a substrate comprising a light absorbing material, the light absorbing material being configured to generate charge carriers upon solar illumination;
an array of nanowires supported by the substrate, each nanowire of the array of nanowires being configured to extract the charge carriers from the substrate, each nanowire of the array of nanowires comprising gallium nitride; and
a plurality of nanoparticles distributed across each nanowire of the array of nanowires, each nanoparticle of the plurality of nanoparticles comprising tin for the catalytic conversion of carbon dioxide (CO₂) in the photoelectrochemical cell into formic acid.

21. The photocathode of claim 20, wherein each nanoparticle of the plurality of nanoparticles comprises tin oxide.

22. A method of fabricating an electrode of an electrochemical system, the method comprising:
growing an array of nanowires on a semiconductor substrate, each nanowire of the array of nanowires having a semiconductor composition for catalytic conversion of carbon dioxide (CO₂) in the electrochemical system; and
depositing a plurality of nanoparticles across each nanowire of the array of nanowires, each nanoparticle of the plurality of nanoparticles having a metallic composition for the catalytic conversion of carbon dioxide (CO₂) in the electrochemical system;
wherein depositing the plurality of nanoparticles comprises implementing a number of electrodeposition cycles, the number of electrodeposition cycles being set such that each nanoparticle of the plurality of nanoparticles has a size at least an order of magnitude smaller than a lateral dimension of each nanowire of the array of nanowires.

23. The method of claim 22, wherein the number of electrodeposition cycles falls in a range from about 60 cycles to about 80 cycles.
